(19) United States
(12) Patent Application Publication
(10) Pub. No.: US 2022/0322997 A1
(43) Pub. Date: Oct. 13, 2022

(54) Wearable Sensor, Perspiration Analysis Device and Method

(71) Applicant: Nippon Telegraph and Telephone Corporation, Tokyo (JP)

(72) Inventors: Yuki Hashimoto, Tokyo (JP); Takako Ishihara, Tokyo (JP); Kei Kuwabara, Tokyo (JP)

(21) Appl. No.: 17/635,777

(22) PCT Filed: Aug. 28, 2019
(86) PCT No.: PCT/JP2019/033651

§ 371 Date: Feb. 16, 2022

(51) Int. Cl. A61B 5/00; A61B 5/145

(52) U.S. Cl. CPC A61B 5/4266; A61B 5/14517; A61B 5/282

(57) ABSTRACT

A wearable sensor includes: a base member that has a through hole serving as a flow path of liquid and a recess connecting with an end portion of the through hole on an outlet side; an electrode disposed on a surface of the base member on which an end portion of the through hole on an inlet side opens; a water absorbing structure disposed on a surface of the base member on the outlet side to come into contact with the liquid flowing into the recess from an opening of the through hole on the outlet side; and an electrode which is water absorbable and disposed on a surface of the water absorbing structure facing the base member to face the opening of the through hole on the outlet side.

### CROSS-REFERENCE TO RELATED APPLICATIONS

This application is a national phase entry of PCT Application No. PCT/JP2019/033651, filed on Aug. 28, 2019, which application is hereby incorporated herein by reference.

### TECHNICAL FIELD

The present invention relates to a wearable sensor for measuring an amount of perspiration, a perspiration analysis device, and a perspiration analysis method.

### BACKGROUND

The human body has tissues that perform electrical activities such as muscles and nerves, and in order to keep these tissues operating normally, it is provided with a mechanism that keeps a concentration of electrolytes in the body constant mainly due to actions of the autonomic nervous system and the endocrine system. For example, when a large amount of water in the body is lost as a result of perspiration due to long-term exposure to a hot environment, excessive exercise, or the like and the concentration of electrolytes in the body deviates from normal values, various symptoms such as heat stroke will occur.

For that reason, monitoring an amount of perspiration can be considered a beneficial method for ascertaining a dehydrated state of the human body.

As a typical method for measuring an amount of perspiration, there is a method for measuring a change in an amount of water vapor during perspiration (see NPL 1). In the method disclosed in NPL 1, an amount of perspiration is estimated based on a difference in humidity with respect to the outside air, and thus the perspiration needs to be vaporized by forced convection using an air pump. In such a method using an air pump, when measurement using a wearable form that a person can wear is considered, the air pump occupies a relatively large volume, and thus there is a problem in reducing the overall size of a device.

### CITATION LIST

### Non Patent Literature

NPL 1: Noriko Tsuruoka, Takahiro Kono, Tadao Matsunaga, Ryoichi Nagatomi, Yoichi Haga, “Development of Small Sweating Rate Meters and Sweating Rate Measurement during Mental Stress Load and Heat Load”, Transactions of Japanese Society for Medical and Biological Engineering, Vol. 54, No. 5, pp. 207-217, 2016.

### SUMMARY

### Technical Problem

Embodiments of the present invention have been made to solve the above problems, and an object thereof is to provide a wearable sensor, a perspiration analysis device, and a perspiration analysis method in which an amount of perspiration can be measured without using an air pump.

### Means for Solving the Problem

A wearable sensor of embodiments of the present invention includes: a base member that has a through hole serving as a flow path of liquid and a first recess connecting with an end portion of the through hole on an outlet side; a first electrode disposed on a surface of the base member on which an end portion of the through hole on an inlet side opens; a water absorbing structure disposed on a surface of the base member on the outlet side to come into contact with the liquid flowing into the first recess from an opening of the through hole on the outlet side; and a second electrode which is water absorbable and disposed on a surface of the water absorbing structure facing the base member to face the opening of the through hole on the outlet side.

Further, a perspiration analysis device of embodiments of the present invention includes the wearable sensor, and a perspiration amount calculation unit configured to calculate an amount of perspiration of a wearer of the wearable sensor based on conduction characteristics between the first and second electrodes due to the perspiration flowing out of the through hole into the first recess and reaching the second electrode.

Also, a perspiration analysis device of embodiments of the present invention includes the wearable sensor, and an electrical resistivity calculation unit configured to calculate electrical resistivity of perspiration of a wearer of the wearable sensor based on conduction characteristics between the first and second electrodes due to the perspiration flowing out of the through hole into the first recess and reaching the second electrode.

Further, a perspiration analysis method of embodiments of the present invention includes: detecting conduction characteristics between a first electrode disposed on a surface of a base member, when a wearable sensor is attached to a body of a wearer, facing a skin of the wearer and a second electrode which is water absorbable and disposed on a surface of a water absorbing structure facing the base member to face an opening of a through hole on an outlet side, the wearable sensor including the base member that has the through hole serving as a flow path of perspiration and a recess connecting with an end portion of the through hole on the outlet side, and a water absorbing structure disposed on a surface of the base member on the outlet side to come into contact with the perspiration flowing into the recess from the opening of the through hole on the outlet side; and calculating an amount of perspiration of the wearer of the wearable sensor based on the conduction characteristics between the first and second electrodes.

### Effects of Embodiments of the Invention

According to embodiments of the present invention, it is possible to measure an amount of liquid flowing into the through hole without using an air pump. Thus, in embodiments of the present invention, when the wearable sensor is attached to the body of the wearer body, the amount of perspiration of the wearer can be measured without using an air pump. In embodiments of the present invention, an air pump is not required, and thus a device can be miniaturized.

### BRIEF DESCRIPTION OF THE DRAWINGS

FIG. 1 is a block diagram illustrating a configuration of a perspiration analysis device according to an embodiment of the present invention.

FIG. 2 is a functional block diagram of an MCU of the perspiration analysis device according to the embodiment of the present invention.

FIG. 3 is a plan view of a wearable sensor of the perspiration analysis device according to the embodiment of the present invention.

FIG. 4 is a cross-sectional view of the wearable sensor of the perspiration analysis device according to the embodiment of the present invention.

FIG. 5 is a cross-sectional view illustrating a state in which perspiration of a wearer flows into a through hole of the wearable sensor in the embodiment of the present invention.

FIG. 6 is an enlarged cross-sectional view of FIG. 5.

FIGS. 7A to 7D are diagrams illustrating an example of change in an electric current value flowing between electrodes in the course of formation and disappearance of droplets of perspiration.

FIG. 8 is a flowchart for explaining an operation of the perspiration analysis device according to the embodiment of the present invention.

FIG. 9 is a block diagram illustrating a configuration example of a computer that realizes the perspiration analysis device according to the embodiment of the present invention.

### DETAILED DESCRIPTION OF ILLUSTRATIVE EMBODIMENTS

### Principles of Embodiments of the Invention

Unlike the method disclosed in NPL 1, embodiments of the present invention are characterized in that an air pump is not required, perspiration is sampled in a liquid state, and an amount of perspiration is measured from a time-series change of an electric current flowing when a voltage is applied to the sampled perspiration. It is also possible to analyze the electrical resistivity of perspiration from the electric current value.

### Embodiments

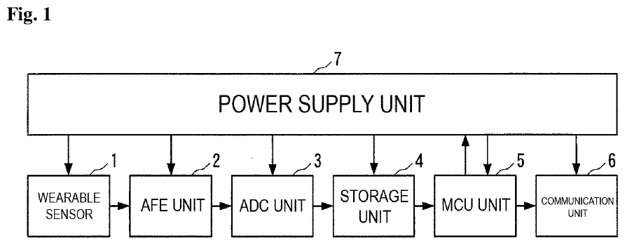

Hereinafter, embodiments of the present invention will be described with reference to the drawings. FIG. 1 is a block diagram illustrating a configuration of a perspiration analysis device according to an embodiment of the present invention. The perspiration analysis device includes a wearable sensor 1, an analog front end (AFE) unit 2, an analog digital converter (ADC) unit 3, a storage unit 4, a micro control unit (MCU) 5, a communication unit 6, and a power supply unit 7.

The wearable sensor 1 detects an electric signal derived from perspiration secreted from a skin of a wearer.

The AFE unit 2 is a circuit that includes an analog front end and amplifies a faint electric signal detected by the wearable sensor 1.

The ADC unit 3 is a circuit that includes an analog to digital converter and converts an analog signal amplified by the AFE unit 2 into digital data at a predetermined sampling frequency.

The storage unit 4 stores the digital data output by the ADC unit 3. The storage unit 4 is realized by a non-volatile memory represented by a flash memory, a volatile memory such as a dynamic random access memory (DRAM), or the like.

The MCU 5 is a circuit that performs signal processing for calculating an amount of perspiration and electrical resistivity of perspiration from the digital data stored in the storage unit 4.

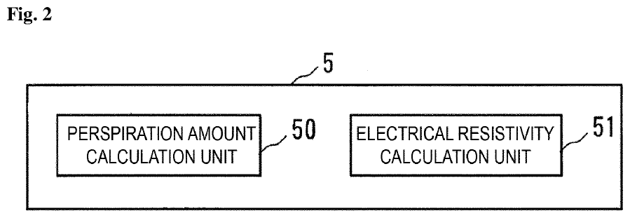

FIG. 2 is a functional block diagram of the MCU 5. The MCU 5 is a circuit that functions as a perspiration amount calculation unit 50 that calculates the amount of perspiration of the wearer based on conduction characteristics between electrodes of the wearable sensor 1, which will be described later, and an electrical resistivity calculation unit 51 that calculates electrical resistivity of the perspiration of the wearer based on the conduction characteristics between the electrodes of the wearable sensor 1.

The communication unit 6 includes a circuit that transmits measurement results and analysis results obtained by the MCU 5 to an external device (not illustrated) such as a smartphone in a wireless or wired manner. Examples of standards for wireless communication include Bluetooth (trade name) Low Energy (BLE) and the like. Further, examples of standards for wired communication include Ethernet (trade name) and the like.

The power supply unit 7 is a circuit responsible for supplying electric power to the perspiration analysis device.

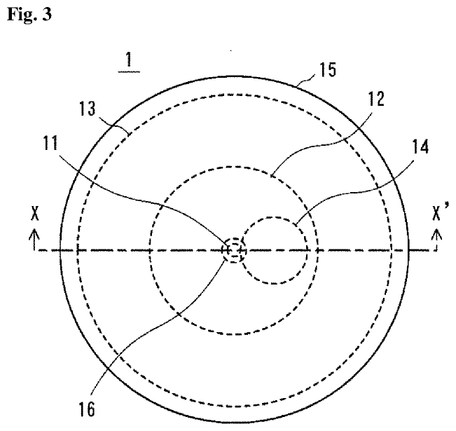

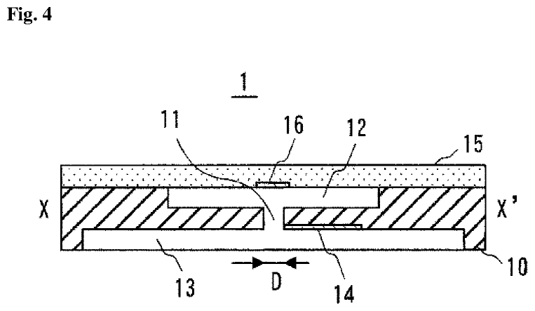

FIG. 3 is a plan view of the wearable sensor 1, and FIG. 4 is a cross-sectional view along line X-X′ of FIG. 3. The wearable sensor 1 includes a base member 10, an electrode 14, a water absorbing structure 15, and a water absorbable electrode 16. The base member 10 has a through hole 11 that serves as a flow path of liquid (perspiration), and a recess 12 that connects with an end portion of the through hole 11 on an outlet side thereof. The electrode 14 is disposed on a surface (lower surface) of the base member 10 on which an end portion of the through hole 11 on an inlet side thereof opens. The water absorbing structure 15 is disposed on a surface (upper surface) of the base member 10 on the outlet side to come into contact with the liquid flowing into the recess 12 from an opening of the through hole 11 on the outlet side. The water absorbable electrode 16 is disposed on a surface of the water absorbing structure 15 facing the base member 10 to face the opening of the through hole 11 on the outlet side.

Examples of the base member 10 include, for example, a base member made of a hydrophilic glass material or a resin material. Also, the base member 10 may be a material subjected to a surface treatment for imparting hydrophilicity to a surface of a water repellent material and an inner surface of the through hole 11. A diameter D of the through hole 11 formed in the base member 10 is, for example, about 1 mm or less.

The recess 12 having a shape in which an upper surface thereof is recessed is formed on the upper surface of the base member 10 to connect with the through hole 11. Conversely, a recess 13 having a shape in which a lower surface thereof is recessed is formed on a lower surface of the base member 10 to connect with the through hole 11.

Also, in embodiments of the present invention, the recess 13 is not an essential constituent requirement. However, in a case in which the recess 13 is provided on a surface (lower surface) of the base member 10 on an inlet side thereof, perspiration can be collected from a wider area of the skin of the wearer skin when the wearable sensor 1 is attached to the body of the wearer body such that the surface of the base member 10 on the inlet side faces the skin of the wearer as described below.

For example, the electrode 14 is made of a metal thin film formed on the surface (lower surface) of the base member 10 on which the end portion of the through hole 11 on the inlet side opens. The electrode 14 is desirably formed in the vicinity of the through hole 11. In the example of FIG. 4, a portion of the electrode 14 is formed to be exposed to the inner surface of the through hole 11, but it may not be exposed.

Examples of the water absorbing structure 15 include fibers such as cotton or silk, a porous ceramic substrate, or the like. Also, the water absorbing structure 15 need not cover the opening of the through hole 11 on the outlet side and the entire surface of the recess 12 and may be disposed to come into contact with a droplet flowing into the recess 12 from the opening of the through hole 11 on the outlet side.

As an example of the electrode 16, a porous metal thin film formed by, for example, plating techniques on the surface of the water absorbing structure 15, an electrode obtained by impregnating the fibers of the water absorbing structure 15 with a conductive polymer, an electrode obtained by weaving conductive fibers (fibers coated with a metal using vapor deposition, etc.) thereinto, or the like can be exemplified.

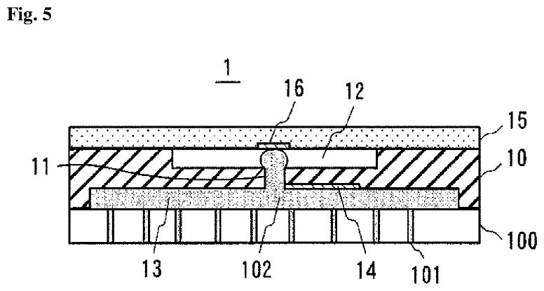

As illustrated in FIG. 5, the wearable sensor 1 is attached to the body of the wearer such that the lower surface of the base member 10 faces the skin of the wearer 100. Reference numeral 101 of FIG. 5 is a perspiration gland of the wearer.

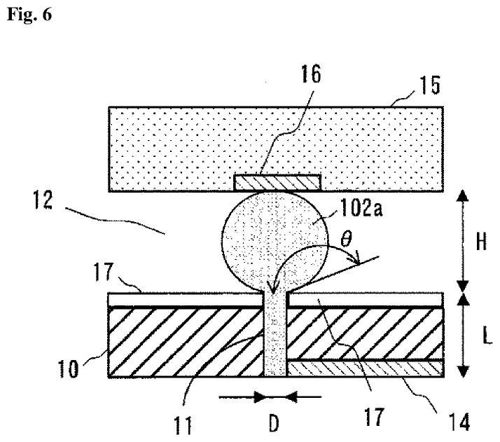

When the wearer perspires, perspiration 102 is introduced into the through hole 11 from inside the recess 13 of the base member 10 due to capillary action. Further, as the amount of perspiration increases, the perspiration 102 rises in the through hole 11 and reaches the recess 12 provided on the upper surface of the base member 10 to connect with the through hole 11 (FIG. 6).

The diameter D of the through hole 11, a length L of the through hole 11, and a hydrophilicity (wettability) of the inner wall of the through hole 11 are set such that the perspiration 102 reaches a position of the recess 12 due to capillary action.

As illustrated in the enlarged view of FIG. 6, a water repellent portion 17 is provided on an inner surface of the recess 12. In a case in which a hydrophilic material is used for the base member 10, the water repellent portion 17 is formed by applying a water repellent surface treatment to the inner surface of the recess 12. In a case in which a water repellent material is used for the base member 10, the water repellent portion 17 can be provided by leaving only the inner surface of the recess 12 as the water repellent material.

When the perspiration 102 reaches the recess 12, it becomes round due to surface tension of the perspiration itself, but its shape varies depending on a state of the recess 12. In the present embodiment, since the water repellent portion 17 is provided on the inner surface of the recess 12, the perspiration 102 reaching the recess 12 becomes a spherical droplet iota as illustrated in FIG. 6. Further, when the amount of perspiration increases, the droplet iota is increased in diameter and finally reaches the electrode 16 and the water absorbing structure 15.

The droplet 102a that has reached the electrode 16 and the water absorbing structure 15 evaporates while moving in the water absorbing structure 15 through a large number of holes in the electrode 16 and a large number of holes in the water absorbing structure 15 due to capillary action. As a result, the droplet 102a disappears.

A distance H between the water repellent portion 17 and the water absorbing structure 15 (a depth of the recess 12) may be set to a value at which the droplet 102a flowing out of the through hole 11 can reach the electrode 16 and the water absorbing structure 15. A fineness of the holes of the electrode 16 and the water absorbing structure 15 and the hydrophilicity (wettability) of the electrode 16 and the water absorbing structure 15 may be set such that the perspiration 102 diffuses to an area on a surface of the wearable sensor 1 opposite to the skin 100 due to capillary action.

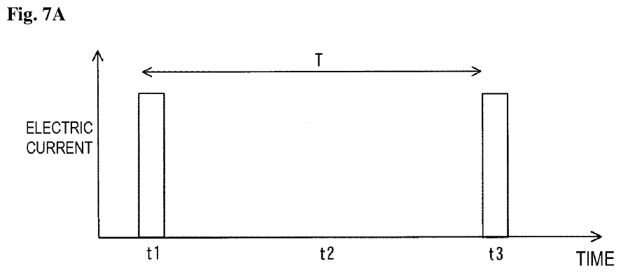

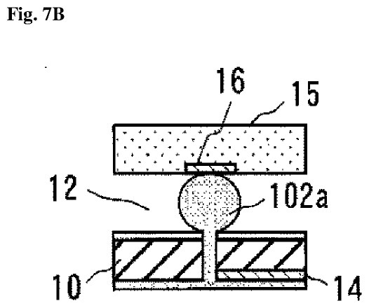

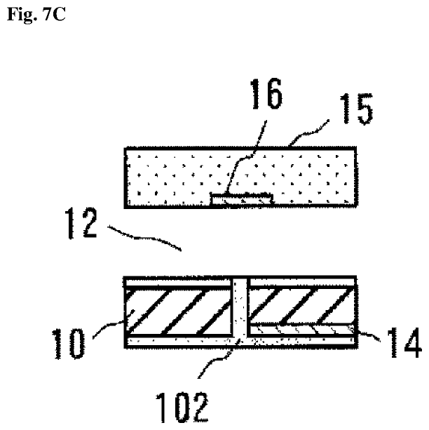

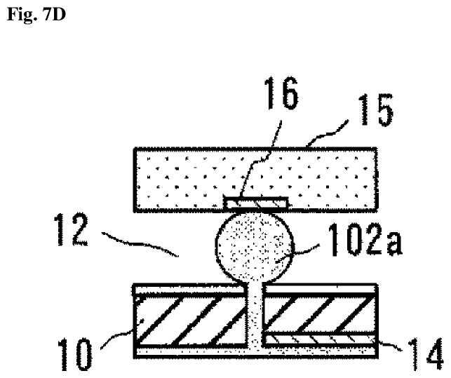

FIG. 7A is a diagram illustrating an example of change in an electric current value flowing between the electrodes 14 and 16 in the course of formation and disappearance of the droplet 102a described above. Also, an electric current waveform illustrated in FIG. 7A is a simplified one of the electric current flowing between the electrodes 14 and 16 and is different from the actual electric current waveform.

At time t1, when the droplet 1o2a of the perspiration 102 comes into contact with the electrode 16 as illustrated in FIG. 7B, the electrodes 14 and 16 become conducting due to the perspiration 102 containing electrolytes, and an electric current flows as shown in FIG. 7A. At time t2, when the droplet 102a disappears as shown in FIG. 7C, no electric current flows. Further, as the amount of perspiration increases, at time t3, when the droplet 102a comes into contact with the electrode 16 as shown in FIG. 7D, the electric current flows again.

In this way, due to the formation and disappearance of the droplet 102a, conduction between the electrodes 14 and 16 is repeatedly generated.

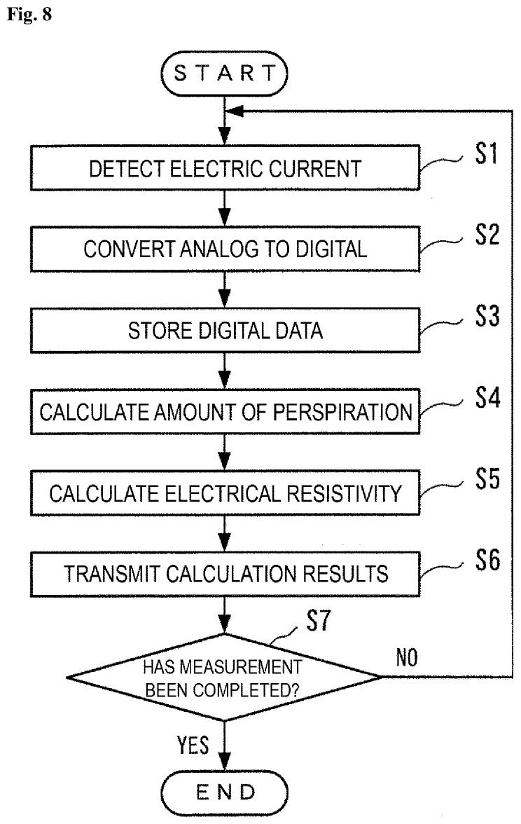

FIG. 8 is a flowchart for explaining an operation of the perspiration analysis device according to the present embodiment. The AFE unit 2 detects the electric current flowing between the electrodes 14 and 16 of the wearable sensor 1 (step S1 in FIG. 8).

The ADC unit 3 converts signals detected and amplified by the AFE unit 2 into digital data (step S2 in FIG. 8). The digital data output from the ADC unit 3 is stored in the storage unit 4 (step S3 in FIG. 8).

The perspiration amount calculation unit 50 calculates the amount of perspiration of the wearer based on the digital data stored in the storage unit 4 (step S4 in FIG. 8). Specifically, the perspiration amount calculation unit 50 sets, as the amount of perspiration, a value obtained by multiplying a volume V of the droplet 102a by the number of times of conduction between the electrodes 14 and 16.

The volume V of the droplet iota can be calculated based on the known distance H between the water repellent portion 17 and the water absorbing structure 15, and an angle (a contact angle θ) formed between a surface of the droplet 102a and a surface of the water repellent portion 17. The contact angle θ can be estimated in advance based on a surface tension of the water repellent portion 17 and a surface tension of the perspiration. Since 99% of the perspiration is water, the surface tension of the perspiration is considered to be dominated by the physical properties of water, and the surface tension of water is defined as the surface tension of the perspiration.

Thus, the volume V of the droplet 102a can be estimated, and the amount of perspiration of the wearer can be estimated.

Further, the perspiration amount calculation unit 50 can calculate a perspiration rate per unit area of the wearer by dividing the volume V of the droplet 102a by a conducting period T (FIG. 7A) between the electrodes 14 and 16, and an area S (an area of the recess 13) of the skin of the wearer 100 covered by the wearable sensor 1.

Also, the electrical resistivity calculation unit 51 calculates an electrical resistivity ρ of the perspiration that changes depending on a concentration of electrolytes in the perspiration of the wearer (step S5 in FIG. 8). Specifically, the electrical resistivity calculation unit 51 calculates a resistance R by dividing a value of a known voltage that the AFE unit 2 applies between the electrodes 14 and 16 by a value of the electric current at the time of conduction indicated by the digital data stored in the storage unit 4. Then, the electrical resistivity calculation unit 51 calculates the electrical resistivity ρ based on the resistance R, the known distance 1 between the electrodes 14 and 16, and a cross-sectional area A of the perspiration between the electrodes 14 and 16. Also, for the cross-sectional area A of the perspiration, a prescribed value may be used when the cross-sectional area of the perspiration between the electrodes 14 and 16 is considered to be constant.

The communication unit 6 transmits the calculation results of the perspiration amount calculation unit 50 and the calculation results of the electrical resistivity calculation unit 51 to an external device (not illustrated) such as a smartphone (step S6 in FIG. 8).

The perspiration analysis device repeatedly performs the processing of steps S1 to S6 until, for example, there is an instruction for measurement completion from the wearer (YES in step S7 in FIG. 8).

As described above, according to the present embodiment, it is possible to realize the measurement of the amount of perspiration of the wearer using a wearable form. In the present embodiment, an air pump is not required, and thus a device can be made smaller than in the method disclosed in NPL 1.

In addition, in the present embodiment, the electrical resistivity of the perspiration of the wearer can be calculated, and it is possible to estimate concentrations of electrolytes (mainly concentrations of Na, K, and Cl) in the perspiration from the electrical resistivity.

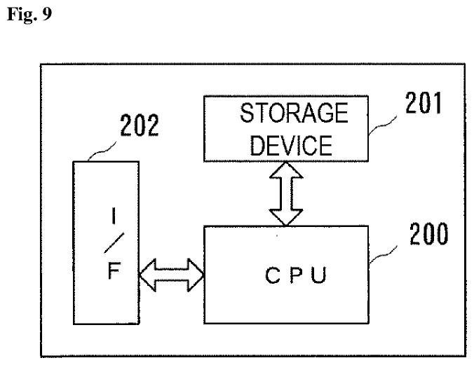

The storage unit 4 and the MCU 5 described in the present embodiment can each be realized by a computer including a central processing unit (CPU), a storage device, and an interface, and programs for controlling these hardware resources. A configuration example of this computer is illustrated in FIG. 9. The computer includes a CPU 200, a storage device 201, and an interface device (hereinafter simply referred to as I/F) 202. The ADC unit 3, the communication unit 6, the power supply unit 7, and the like are connected to the I/F 202. In such a computer, a program for realizing the perspiration analysis method of embodiments of the present invention is stored in the storage device 201. The CPU 200 executes the processing described in the present embodiment in accordance with the program stored in the storage device 201.

### INDUSTRIAL APPLICABILITY

The present invention can be applied to a technique for analyzing an amount of perspiration.

### REFERENCE SIGNS LIST

1 Wearable sensor

2 AFE unit

3 ADC unit

4 Storage unit

5 MCU

6 Communication unit

7 Power supply unit

10 Base member

11 Through hole

12, 13 Recess

14, 16 Electrode

15 Water absorbing structure

17 Water repellent portion

50 Perspiration amount calculation unit

51 Electrical resistivity calculation unit.

1.-8. (canceled)

9. A wearable sensor, comprising:
a base member that includes a through hole serving as a flow path of liquid and a first recess on a first side of the base member, the first recess being connected to a first end portion of the through hole on the first side;
a water absorbing structure disposed on a first surface of the base member, the water absorbing structure being configured to come into contact with the liquid flowing into the first recess from a first opening of the through hole on the first side;
a first electrode disposed on a second surface of the base member on which a second end portion of the through hole is disposed; and
a second electrode which is water-absorbable and disposed on a surface of the water absorbing structure facing the base member, the second electrode facing the first opening of the through hole.

10. The wearable sensor according to claim 9, wherein a second side of the base member faces a skin of a wearer when the base member is attached to a body of the wearer to face the skin of the wearer.

11. The wearable sensor according to claim 10, wherein an electrical resistivity calculator is configured to calculate an electrical resistivity of perspiration of the wearer of the wearable sensor based on conduction characteristics between the first electrode and second electrode due to the perspiration flowing out of the through hole into the first recess and reaching the second electrode.

12. The wearable sensor according to claim 9, wherein a second surface of the base member and an inner surface of the through hole have hydrophilicity, and wherein an inner surface of the first recess has water repellency.

13. The wearable sensor according to claim 9, wherein the base member further includes a second recess provided on the second surface, the second recess being connected to the second end portion of the through hole.

14. A perspiration analysis device, comprising:
a wearable sensor comprising:
a base member that includes a through hole serving as a flow path of liquid and a first recess on a first side of the base member, the first recess being connected to a first end portion of the through hole on the first side;
a water absorbing structure disposed on a first surface of the base member, the water absorbing structure being configured to come into contact with the liquid flowing into the first recess from a first opening of the through hole on the first side;
a first electrode disposed on a second surface of the base member on which a second end portion of the through hole is disposed; and
a second electrode which is water-absorbable and disposed on a surface of the water absorbing structure facing the base member, the second electrode facing the first opening of the through hole, wherein a second side of the base member faces a skin of a wearer when the base member is attached to a body of the wearer to face the skin of the wearer; and
a perspiration amount calculator configured to calculate an amount of perspiration of a wearer of the wearable sensor based on conduction characteristics between the first and second electrodes due to the perspiration flowing out of the through hole into the first recess and reaching the second electrode.

15. The perspiration analysis device according to claim 14,
wherein the perspiration amount calculator is configured to set, as the amount of perspiration, a value obtained by multiplying a presumed volume of a droplet of the perspiration that has flowed out of the through hole into the first recess and reached a height reaching the second electrode by a number of times of conduction between the first and second electrodes.

16. The perspiration analysis device according to claim 14, wherein a second surface of the base member and an inner surface of the through hole have hydrophilicity, and wherein an inner surface of the first recess has water repellency.

17. The perspiration analysis device according to claim 14, wherein the base member further includes a second recess provided on the second surface, the second recess being connected to the second end portion of the through hole.

18. A perspiration analysis method, comprising:
providing a wearable sensor including:
a base member that has a through hole serving as a flow path of perspiration and a recess connected to an end portion of the through hole; and
a water absorbing structure disposed on a surface of the base member coming into contact with the perspiration flowing into the recess from an opening of the through hole;
detecting conduction characteristics between a first electrode disposed on a surface of a base member facing, when the wearable sensor is attached to a body of a wearer, a skin of the wearer and a second electrode which is water-absorbable and disposed on a surface of the water absorbing structure facing the opening of the through hole; and
calculating an amount of perspiration of the wearer of the wearable sensor based on the conduction characteristics between the first electrode and the second electrode.

19. The perspiration analysis method according to claim 18, wherein a second surface of the base member and an inner surface of the through hole have hydrophilicity, and wherein an inner surface of the recess has water repellency.

20. The perspiration analysis method according to claim 18, wherein the base member further includes a second recess provided on a second surface of the base member on which a second end portion of the through hole is disposed, the second recess being connected to the second end portion of the through hole.
